(19) United States
(12) Patent Application Publication
(10) Pub. No.: US 2020/0171370 A1
(43) Pub. Date: Jun. 4, 2020

(54) MULTI-PARAMETER PERFORMANCE ASSESSMENT AND CONDITIONING SYSTEM

(71) Applicant: David Clarke, Miami Beach, FL (US)

(72) Inventor: David Clarke, Miami Beach, FL (US)

(21) Appl. No.: 16/208,959

(22) Filed: Dec. 4, 2018

(51) Int. Cl. A63B 71/06; A61B 5/0476; A61B 5/0488; A61B 5/00; A61B 5/16

(52) U.S. Cl. CPC A63B 71/06; A61B 5/0476; A61B 5/0488; A61B 5/6814; A61B 5/165; A61B 5/0002; A61B 5/7445; A61B 5/7475; A61B 5/6823

(57) ABSTRACT

The multi-parameter performance assessment and conditioning system disclosed herein is designed to assess and condition the peak performance of an athlete by measuring various biophysical parameters and employing a proprietary software algorithm to produce a real-time assessment score. The score can be monitored by the athlete in real time, by a proprietary software on a computing system or on a wrist band, so that the user is aware of the effect that their body's behavior is having on their performance. The system comprises an electroencephalogram, an electromyogram, and a series of physiological monitors to track the user's biophysical parameters, and can be implemented in a static training environment, a kinetic training environment, or on the user's field of play.

### TECHNICAL FIELD OF THE INVENTION

The present invention relates in general to physiological assessment devices, and, more specifically, to a multi-parameter performance assessment and conditioning system.

### BACKGROUND OF THE INVENTION

Physiological stress is an organism's response to a stressor, such as an environmental condition or a particular situation that involves triggering the autonomic nervous system and the hypothalamic-pituitary-adrenal axis. A common manifestation of stress is the fight-or-flight response, which involves a hormonal cascade that results in the discharge of epinephrine and norepinephrine, and which triggers a number of physical changes such as increased heart rate, increased rate of breathing, pupillary dilation, loss of peripheral vision, and shaking of the muscles. Stress management is a wide spectrum of techniques and physiotherapies aimed at controlling a person's level of stress and anxiety, and reactions to stressful situations.

The physiological and biological changes associated with such reactions are well-known and measureable using a variety of devices, depending on the parameters to be measured. Most commonly, obvious physical changes, such as those related to blood pressure and heart rate may be measured using such devices as a sphygmomanometer, a heart rate monitor, or other physiological monitors. Less obvious physical changes, such as those associated with the electrical activity produced by skeletal muscles, may also be measured using such devices as an electromyogram (EMG), which detects the electrical potential generated by muscle cells when these cells are electrically or neurologically activated. Even the changes in brain wave activity associated with stress may be measured using such devices as an electroencephalogram (EEG), which measures voltage fluctuations resulting from ionic current within the neurons of the brain.

Athletes, especially professional athletes, are expected to operate at elevated performance levels, and their training often involves repetition of physical activities and behaviors common to their sport. Such training may incorporate physiological measurement devices for assessment of physical performance and changes in performance or recovery from injury, though the assessment of stress and development of stress management techniques occurs only incidentally to this training. The physical performance of an athlete is strongly correlated with his or her ability to manage their physiological parameters, and the management of stress, which has a significant effect on these parameters, is a vital aspect of assessing and improving athletic performance.

Thus, there is a need in the art for a multi-parameter performance assessment and conditioning system that assesses the biological parameters associated with stress and physical performance, and provides a training and reassessment protocol for conditioning such physical performance and managing stress levels.

### BRIEF SUMMARY OF THE INVENTION

To minimize the limitations in the prior art, and to minimize other limitations that will be apparent upon reading and understanding the present specification, the present invention describes a multi-parameter performance assessment and conditioning system.

It is an objective of the present invention to provide an assessment and conditioning system that may be implemented on a computing device.

It is another objective of the present invention to provide an assessment and conditioning system that may comprise a proprietary software.

It is another objective of the present invention to provide an assessment and conditioning system that may comprise a central database.

It is another objective of the present invention to provide an assessment and conditioning system that may comprise a user database.

It is another objective of the present invention to provide an assessment and conditioning system that may comprise an electroencephalogram.

It is another objective of the present invention to provide an assessment and conditioning system that may comprise an electromyogram.

It is another objective of the present invention to provide an assessment and conditioning system that may comprise a physiological monitor.

It is another objective of the present invention to provide an assessment and conditioning system that may comprise a plurality of wearable sensors.

It is another objective of the present invention to provide an assessment and conditioning system that may comprise a static training environment.

It is another objective of the present invention to provide an assessment and conditioning system that may comprise a kinetic training environment.

It is another objective of the present invention to provide an assessment and conditioning system that may comprise a performance assessment score.

It is another objective of the present invention to provide an assessment and conditioning system that may comprise a wearable performance reporting device.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 illustrates an overview of a multi-parameter performance assessment and conditioning system static training environment, as contemplated by the present disclosure;

FIG. 2 illustrates an overview of a multi-parameter performance assessment and conditioning system static training environment, as contemplated by the present disclosure;

FIG. 3 illustrates an overview of a multi-parameter performance assessment and conditioning system static training environment, as contemplated by the present disclosure;

FIG. 4 illustrates a user video interface of a multi-parameter performance assessment and conditioning system static training environment, as contemplated by the present disclosure;

FIG. 5 illustrates a plurality of user input devices of a multi-parameter performance assessment and conditioning system static training environment, as contemplated by the present disclosure;

FIG. 6 illustrates an electroencephalogram component of a multi-parameter performance assessment and conditioning system, as contemplated by the present disclosure;

FIG. 7 illustrates an electroencephalogram component of a multi-parameter performance assessment and conditioning system, as contemplated by the present disclosure;

FIG. 8 illustrates a virtual reality display of a multi-parameter performance assessment and conditioning system, as contemplated by the present disclosure; and

FIG. 9 schematically presents a computing system configured to carry out and actualize methods and tasks described herein, as contemplated by the present disclosure.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

The present invention relates in general to physiological assessment devices, and, more specifically, to a multi-parameter performance assessment and conditioning system. As contemplated by the present disclosure, the system may comprise a proprietary software on a computing device, a plurality of physiological monitors, and a training environment. The proprietary software may receive inputs from the plurality of physiological monitors and may determine a plurality of assessment scores based on such inputs. The receiving of inputs may occur in real-time or may be recorded for later playback and review. The training environment may comprise a static training environment or may comprise a kinetic training environment, and the specific composition of the training environment may vary as required for different training needs.

In more detail, the plurality of physiological monitors may comprise an electroencephalogram (EEG), an electromyogram (EMG), and any other appropriate physiological monitors sufficient to acquire the biological parameters monitored by the system. Such biological parameters may include, though are not limited to, brainwave patterns, muscle function, body temperature, sweat levels, heart rate, respiratory rate, blood oxygen levels, pupillary dilation, and any other parameters able to be acquired non-invasively. In an alternative embodiment the system may further comprise physiological monitors capable of acquiring parameters invasively.

The proprietary software of the system may receive these plurality of biological parameters from the various physiological monitors in real time, and may implement a proprietary algorithm by which to calculate a series of standardized scores. A single, overall assessment score may be calculated via the proprietary algorithm, along with multiple sub-scores to emphasize major categories of focused assessment. These various calculated scores may then be presented to a user of the system in real time, may be presented in real time to a third party monitoring the system, or may be recorded for later review. The various calculated scores may vary significantly during the course of a user session as they are intended to display and assess a user's performance at any given moment. The recorded scores may be recorded over time, and may be displayed as a progression of score changes over time and in association with notable or marked events.

By way of example, an athlete user who may be a basketball player may be equipped with the EEG, EMG, and physiological monitors of the system while at rest, and the system may calculate a baseline score for that user. The user may then begin to perform tasks common to the sport of basketball such as, for example, shooting free throws or dribbling up and down the court. The various physiological monitors of the system may identify and report changes in the user's biological parameters while the user is conducting these exercises so that the user may also have real time score interpretations of their performance during the exercise. It is contemplated that the various physiological monitors may be hardwired to the system or may be wirelessly connected to the system, so that a user may engage in more strenuous or wide-ranging activities while still being monitored.

The result of such monitoring is that the user may be given a record of biophysical changes that have occurred during their performing of tasks common to their sport. The identification of various parameters at various stages of exercise may grant to the user areas of focus in which to improve their performance, and may allow the user to identify potential weaknesses or problem areas in their physical performance.

The exercises performed by the user may be performed in any appropriate environment. As contemplated by the present disclosure, the user may perform exercises within the play area appropriate to their sport, within a kinetic training environment, or within a static training environment.

In one embodiment, the kinetic training environment may be a dedicated area in which the user may practice the motions and behaviors common in their sport while being monitored by the system. By way of example, an athlete user who may be a golf player may be equipped with the EEG, EMG, and physiological monitors of the system and may be placed within a kinetic training environment that comprises an indoor golf simulator. An athlete user who may be a baseball player may be similarly equipped and placed within a kinetic training environment that comprises an indoor baseball simulator. Within such environments the user may then perform activities that relate to their sport and may receive real time scoring related to their performance.

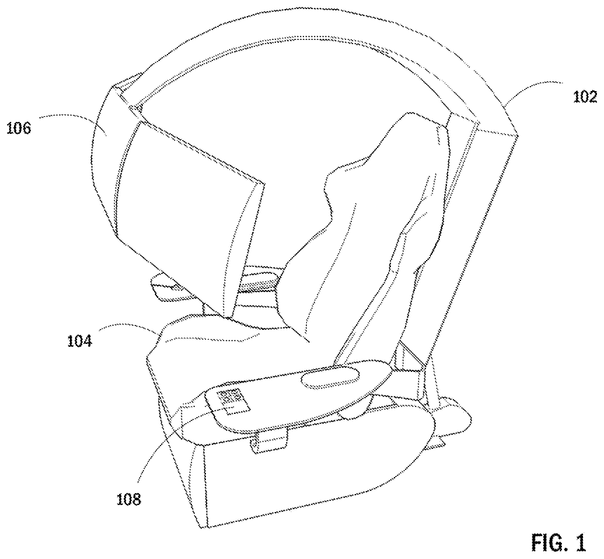

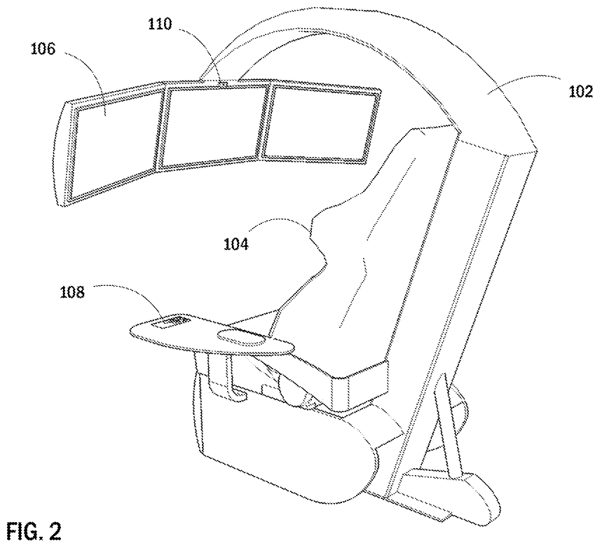

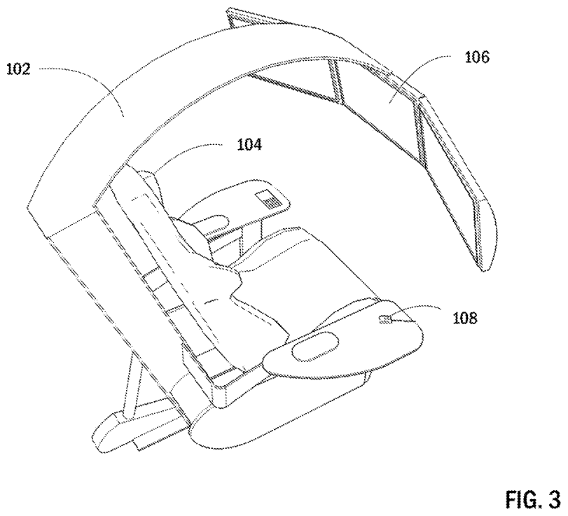

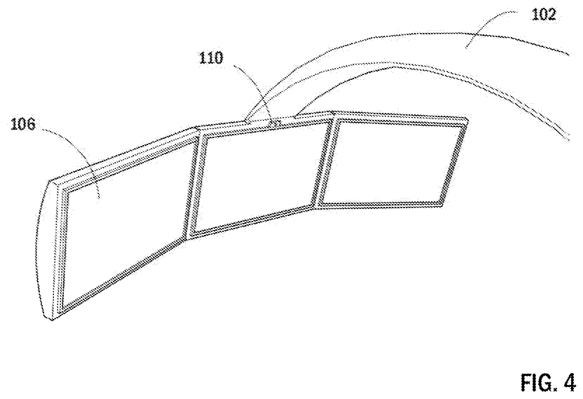

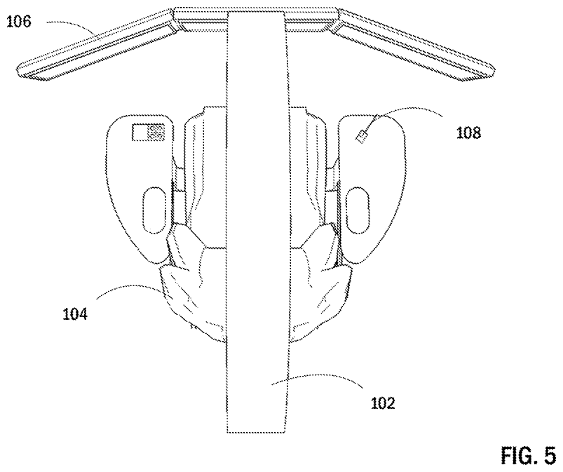

The illustrations of FIGS. 1-5 illustrate a multi-parameter performance assessment and conditioning system static training environment. In one embodiment the static training environment may comprise a frame 102, a chair 104, a display output 106, a physical input 108, and a visual input 110. The static training environment may be a dedicated area in which the user may practice stationary exercises designed to assess or enhance the user's performance. An athlete user may be equipped with the EEG, EMG, and physiological monitors of the system and may begin by sitting in the chair 104 and engaging the system. An onboard computing system 900 may be installed within the frame 102, or the plurality of monitors may be wirelessly connecting to a computing system 900 remote from the static training environment. The user may be shown one or more exercises via the display output 106, and may interact with these exercises via the physical inputs 108 and the visual input 110. The visual input 100 may be equipped with facial recognition technology and may also act as a physiological monitor within the system. The static training environment may further comprise a plurality of additional functions, such as, for example, sound output, vibrational output, sound input, heat sensors, sweat sensors, or any other appropriate input or output devices.

The display output 106 may be any appropriate visual display output. In one embodiment the display output 106 may comprise a plurality of display monitors allowing the user to view system status, exercise status, the exercise itself, their current assessment score, their selected biographical information, and any other appropriate data simultaneously. In another embodiment the display output 106 may comprise a display monitor integrated into a mobile device, such as a tablet or smartphone, or a wearable device, such as a smart wrist band, which allows the user to view such information remote from the static training environment.

The physical input 108 may be any appropriate physical input. In one embodiment the physical input 108 may comprise a mouse and keyboard combination for interfacing with an onboard or remote computing system 900. In a second embodiment the physical input 108 may comprise a tablet device wirelessly connected to the computing system 900.

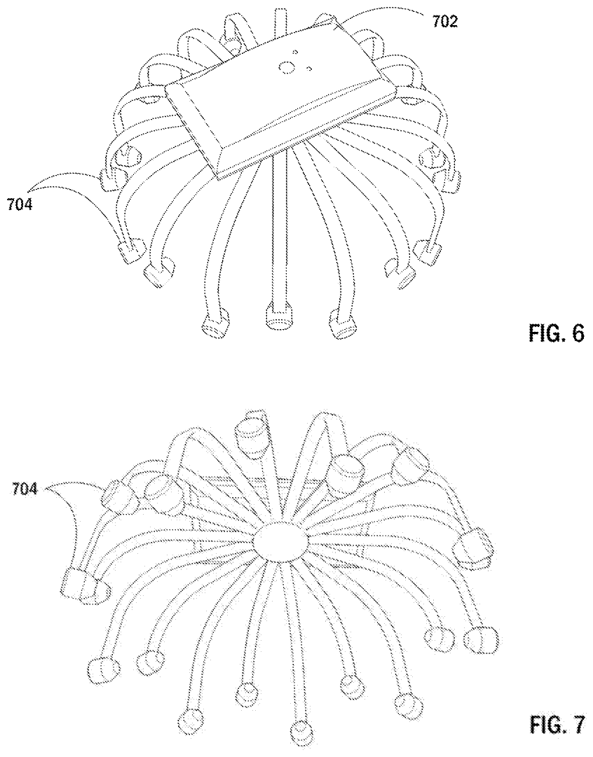

The illustrations of FIGS. 6 and 7 illustrate an electroencephalogram component of a multi-parameter performance assessment and conditioning system. In one embodiment of the system, the EEG device, which may read brainwave patterns, may comprise a control unit 702 and a plurality of sensors 704, and is worn on the user's head. The EEG may also comprise a combination device that reads, along with brainwave activity, any parameters accessible from the user's head such as body temperature, sweat levels, pupillary dilation, pulse, or breathing rate. To effect such functions the control unit 702 may comprise a central processor receiving input signals from the plurality of sensors 704 and relaying these input signals to the proprietary software of the computing system 900. The connection between the control unit 702 and the computing system 900 may be a wired connection or may be a wireless connection. The plurality of sensors 704 may be any sensors appropriate to the desired readings, such as, for example, EEG sensors, temperature sensors, sweat sensors, pupil dilation sensors, heart rate monitors, or any combination thereof.

The EEG may exist as a standalone device or may be installed into a hat or helmet of an athlete user. By way of example, the EEG device may be installed into the batting helmet of a baseball user so that the user may receive monitoring and assessment scores while practicing their exercises in full uniform. The EEG device may be similarly installed into a football or hockey helmet so that such users may receive monitoring and assessment scores while exercising within the play area appropriate to their sport. In one embodiment of the system the plurality of monitors may be worn by a user during competitive playing of their sport so that their game time performance may be monitored and assessed.

In one embodiment of the system, the EMG device, which may read muscle function, may comprise a control unit and a plurality of sensors, and may be worn on any part of the user's body. The control unit may comprise a central processor receiving input signals from the plurality of sensors and relaying these input signals to the proprietary software of the computing system 900. The connection between the control unit and the computing system 900 may be a wired connection or may be a wireless connection. By way of example, the EMG device may be integrated into a vest for wearing on a user's torso. In such a form the EMG device may receive input signals from the various chest, abdominal, and back muscles of the user during exercise. Such an EMG device may further incorporate sensors for measuring any biophysical parameters appropriate to measure from the user's torso, such as, for example, heart rate, body temperature, sweat, respiratory rate, or any other appropriate parameter. In one embodiment the EMG device may comprise a sleeve for wearing on a user's arm, or may comprise a legging for wearing on a user's leg, and may measure skeletal muscle signals from these areas along with body temperature, sweat levels, blood pressure, or any other appropriate parameters.

In one embodiment of the system, the proprietary software may further comprise a mobile application software that may be downloaded and installed onto a mobile device, such as a smartphone, tablet, Apple watch, or other similar device. The application may be wirelessly connected to the plurality of physiological monitors being worn by a user, and may be further wirelessly connected to the central database of the system. The user may perform the exercises and assessments related to their sport while wearing the plurality of physiological monitors, and may then receive feedback and score reporting from the proprietary software via the mobile application. In this way, the user is able to monitor their progress and assessment score in real time, regardless of their proximity to a fixed computing device or the static training environment. A wearable device, such as a smart wrist band or an Apple watch, is considered particularly useful to achieve this purpose in the kinetic training environment and during game time performance.

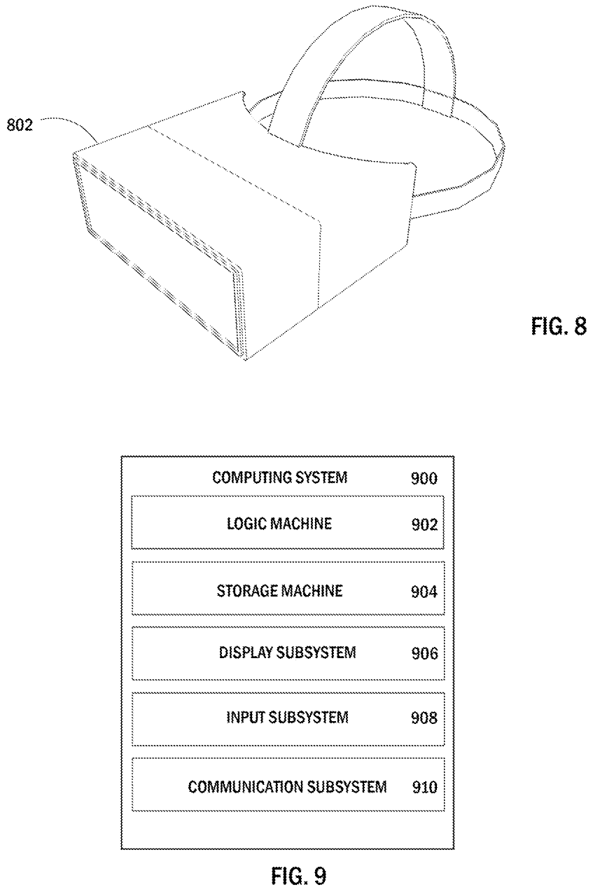

The illustration of FIG. 8 illustrates a virtual reality display of a multi-parameter performance assessment and conditioning system static training environment. The virtual reality display 802 may be worn by a user to provide additional functionality and conditioning during various exercises in the kinetic training environment or static training environment, as desired. By way of example, where an athlete user is travelling for an away game and does not have ready access to the various environments of the system, the user may continue to practice their exercises remotely using the virtual reality display. By way of a second example, the virtual reality display may augment the experience of the kinetic training environment by presenting training scenarios not otherwise reproducible within the parameters of the environment.

The illustration of FIG. 9 schematically presents a computing system that may represent an embodiment of the present invention. In some embodiments the method is executed on a computing system such as computing system 900 of FIG. 9. For example, storage machine 904 may hold instructions executable by logic machine 902 to provide the method to users. Display subsystem 906 may display the various elements of the method to participants. For example, display subsystem 906, storage machine 904, and logic machine 902 may be integrated such that the method may be executed while being displayed on a display screen. The input subsystem 908 may receive user input from participants to indicate the various choices or user inputs described above. The described method may be executed, provided or implemented to a user on one or more computing devices via a computer-program product such as via an application programming interface (API). FIG. 9 schematically shows a non-limiting exemplary embodiment of a computing system 900 that can enact the method described above. Computing system 900 may be any appropriate computing device such as a personal computer, tablet computing device, gaming device or console, mobile computing device, etc. Computing system 900 includes a logic machine 902 and a storage machine 904. Computing system 900 may include a display subsystem 906, input subsystem 908, and communication subsystem 910. Logic machine 902 may execute machine-readable instructions via one or more physical devices. For example, the logic machine 902 may be configured to execute instructions to perform tasks for a computer program. The logic machine may include one or more processors to execute machine-readable instructions. Storage machine 904 includes one or more physical devices configured to hold or store instructions executable by the logic machine to implement the method. When such methods and processes are implemented, the state of storage machine 904 may be changed to hold different data. For example, storage machine 904 may include memory devices such as various hard disk drives or CD or DVD devices. Display subsystem 906 may visually present data stored on storage machine 904. For example, display subsystem 906 may visually present data to form a graphical user interface (GUI). Input subsystem 908 may be configured to connect and receive input from devices such as a mouse, keyboard, or gaming controller. Communication subsystem 910 may be configured to enable system 900 to communicate with other computing devices. Communication subsystem 910 may include wired and/or wireless communication devices to facilitate networked communication.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

I claim:

1. An assessment and conditioning system, comprising:
a wearable EEG;
a wearable EMG;
a proprietary software; and
a display output;
wherein said wearable EEG comprises an article worn on a head of a user and integrates a plurality of electroencephalographic sensors and a wireless connectivity;
wherein said wearable EMG comprises an article worn on an upper torso of a user and integrates a plurality of electromyographic sensors, a plurality of physiological sensors, and a wireless connectivity;
wherein said plurality of electroencephalographic sensors detect a brainwave pattern;
wherein said plurality of electromyographic sensors detect a muscular pattern;
wherein said plurality of physiological sensors detect a physiological pattern;
wherein said detected brainwave pattern, said detected muscular pattern, and said detected physiological pattern are transmitted via said wireless connectivity to said proprietary software;
wherein said proprietary software receives said detected brainwave pattern, said detected muscular pattern, and said detected physiological pattern;
wherein said detected brainwave pattern, said detected muscular pattern, and said detected physiological pattern are processed by said proprietary software using a proprietary algorithm to determine a plurality of assessment scores; and
wherein said proprietary software transmits said plurality of assessment scores to said display output for display.

2. The invention of claim 1,
wherein said plurality of assessment scores include an overall score, a plurality of category scores, a base line score, and a current score;
wherein said plurality of assessment scores are digitally stored in a score history file;
wherein said plurality of assessment scores digitally stored in said score history file may be compared against one another by said proprietary software to determine a user progress report; and
wherein said proprietary software transmits said user progress report to said display output for display.

3. The invention of claim 2,
wherein said display output is a mobile computing device.

4. The invention of claim 3,
wherein said mobile computing device is a smart device worn on a wrist of said user.

5. The invention of claim 4,
wherein said mobile computing device receives a plurality of system commands from said user; and
wherein said plurality of system commands act as operating instructions for said proprietary software.

6. The invention of claim 2, further comprising:
a static environment;
wherein said static environment comprises a sitting area.

7. The invention of claim 6, further comprising:
a static environment frame;
a plurality of input devices;
a plurality of output devices;
a plurality of monitors;
an onboard computing device; and
a wireless connectivity;
wherein said sitting area, said plurality of input devices, said plurality of output devices, said plurality of monitors, and said onboard computing device are installed on said static environment frame.

8. The invention of claim 7,
wherein said plurality of input devices comprise a mouse and a keyboard;
wherein said plurality of output devices comprise a speaker;
wherein said plurality of monitors comprise said display output;
wherein said mouse and said keyboard receive a plurality of system commands from said user; and
wherein said plurality of system commands act as operating instructions for said proprietary software.

9. The invention of claim 8,
wherein said plurality of input devices further comprise a tablet device;
wherein said tablet device receives a plurality of system commands from said user; and
wherein said plurality of system commands act as operating instructions for said proprietary software.

10. The invention of claim 2, further comprising:
a kinetic environment;
wherein said kinetic environment comprises a simulation area.

11. The invention of claim 10, further comprising
a virtual reality display;
wherein said virtual reality display comprises an article worn on a head of a user.

12. The invention of claim 10,
wherein said simulation area is a physical sports simulator.

13. The invention of claim 10,
wherein said simulation area is a virtual sports simulator.

14. An assessment and conditioning system, comprising:
a wearable EEG;
a wearable EMG;
a proprietary software; and
a display output;
wherein said wearable EEG further comprises a plurality of electroencephalographic sensors;
wherein said wearable EMG further comprises a plurality of electromyographic sensors and a plurality of physiological sensors;
wherein said plurality of electroencephalographic sensors detect a brainwave pattern;
wherein said plurality of electromyographic sensors detect a muscular pattern;
wherein said plurality of physiological sensors detect a physiological pattern;
wherein said detected brainwave pattern, said detected muscular pattern, and said detected physiological pattern are transmitted to said proprietary software;
wherein said proprietary software receives said detected brainwave pattern, said detected muscular pattern, and said detected physiological pattern;
wherein said detected brainwave pattern, said detected muscular pattern, and said detected physiological pattern are processed by said proprietary software using a proprietary algorithm to determine a plurality of assessment scores; and
wherein said proprietary software transmits said plurality of assessment scores to said display output for display.

15. The invention of claim 14,
wherein said wearable EEG is worn on a head of a user; and
wherein said wearable EMG is worn on a body of said user.

16. The invention of claim 15,
wherein said plurality of assessment scores include an overall score, a plurality of category scores, a base line score, and a current score;
wherein said plurality of assessment scores are digitally stored in a score history file;
wherein said plurality of assessment scores digitally stored in said score history file may be compared against one another by said proprietary software to determine a user progress report; and
wherein said proprietary software transmits said user progress report to said display output for display.

17. The invention of claim 16,
wherein said display output is a mobile computing device.

18. The invention of claim 17,
wherein said mobile computing device receives a plurality of system commands from said user; and
wherein said plurality of system commands act as operating instructions for said proprietary software.

19. The invention of claim 18,
wherein said mobile computing device is a smart device worn on a wrist of said user.
